(19) United States
(12) Patent Application Publication
(10) Pub. No.: US 2009/0292366 A1
(43) Pub. Date: Nov. 26, 2009

(54) Material based on a partially stabilized zirconia matrix with cerium oxide and yttrium oxide as stabilizing oxides of zirconia, a process for preparation and use of the material

(76) Inventors: Wolfgang Burger, Plochingen (DE); Wolfgang Leonhardt, Hochdorf (DE); Norbert Thiel, Bad Sackingen (DE); Marc Stephan, Lorrach (DE)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 12/311,590

(22) PCT Filed: Oct. 5, 2007
(86) PCT No.: PCT/EP2007/060619

§ 371 Date: Jul. 24, 2009

Related U.S. Application Data
(60) Provisional application No. 60/849,467, filed on Oct. 5, 2006.

(30) Foreign Application Priority Data
Oct. 5, 2006 (EP) 06121793.1

(51) Int. Cl. A61F 2/28; C04B 35/48; C04B 35/64

(52) U.S. Cl. 623/23.56; 501/105; 264/604

(57) ABSTRACT

A sintered ceramic material comprising: a) from 98-50% by volume of zirconia as a matrix, i) stabilized with a stabilizing composition having ii) of from about 2 to about 3 mole percent of yttria and of from about 10 to about 15 mole percent of ceria; wherein the term mole percent is related to the zirconia matrix and iii) the stabilizing composition is present in the range of from about 1:99 to about 99:1; and b) from about 2 to about 50% by volume of alumina of which from about 5 to about 90% by volume is in the form of hexagonal platelets of general formula REAl 11018, where RE stands for rare earth metal.

The present invention relates to a material based on a partially stabilized zirconia matrix, and to a process for the preparation and use of the material. The material according to the invention can be employed, for example, as a sintered compact for various fields of application.

Tetragonally stabilized zirconia materials are known in the prior art. They usually have a high mechanical strength and a relatively high fracture toughness. In addition, they are biocompatible.

As a disadvantage of these materials, their relatively low hydrothermal resistance has been found. In a humid atmosphere, the materials lose strength. A number of attempts have been made already in the prior art to improve their hydrothermal resistance. Thus, a significantly improved hydrothermal resistance from the alloying of alumina in concentrations of less than 0.5% by weight and applying sinter temperatures of 1350° C. over that of conventionally prepared tetragonally stabilized zirconia has been published in the form of a product data sheet (TOSOH ZIRCONIA POWDER “E” GRADES—new improved zirconia powder; printed April 3 in Japan).

In an earlier work, the coating of the zirconia grains with the stabilizing yttrium oxide has been described, and an amount of 0.1% by weight of alumina was already contained in this composition (W. Burger et al., Journal of Materials Science: Materials in Medicine 8 (1997) 113-118; C. Piconi et al., Biomaterials 19 (1998) 1489-1494). It was attempted to create improved materials.

Further, EP-A-0 466 836 relates to reinforcement of ceramic materials with platelets. This document relates to a ceramic body consisting of from 10 to 99% by volume of a zirconia matrix that is partially stabilized and from 1 to 90% by volume of SrAl₁₂O₁₉ platelets with an aspect ratio of >2. The molar ratio of SrO:Al₂O₃ is specified to be 0.01 or 0.02 to 0.2 or 0.3. In the stoichiometric composition, the ratio of SrO:Al₂O₃=1:6=0.17. Thus, alumina and zirconia may be in excess.

EP-A-0 542 815 relates to a sintered molding consisting of a matrix material formed from an alumina/chromium oxide mixed crystal and embedded into the zirconia. As stabilizing oxides, oxides of cerium, praseodymium, terbium or yttrium are employed. The stabilizing oxides are added in such amounts that more than 90% by volume of the zirconia is in the tetragonal modification. The molar ratio between the zirconia containing the stabilizing oxides and chromium oxide is from 1000:1 to 20:1.

In particular, a material is described that comprises a matrix whose proportion is 60 to 98% by volume and which consists of 67.1% by volume of an Al₂O₃—Cr₂O₃ mixed crystal and from 0.8 to 32.9% by volume of hexagonal SrAl₁₂₋ₓCrₓO₁₉ platelets as well as 2-40% by volume of tetragonally stabilized zirconia. DE-A-198 50 366 relates to a sintered compact with a matrix material that contains, in addition to an alumina/chromium oxide mixed crystal, another mixed crystal selected from at least one mixed crystal according to the general formulas stated therein and contains alkali metals, alkaline earth metals, cadmium, lead or mercury and rare earth metals.

M. Miura, H. Hongoh, T. Yogo, S. Hirano and T. Fujii disclose in “Formation of plate like lanthanum-β-Aluminate crystal in Ce-TZP matrix” (J. Mat. Sci., 29 (1994), 262-268) besides a material system Ce-TZP/Al₂O₃/La₂O₃ the influence of grain size of used aluminium oxide particles on platelet formation when using very fine powders. It was found a rather independent size of the platelets having a rather course grain size in the matrix independent of the sinter temperature used. Formation of platelets has been monitored beyond 1.500° C. and the complete formation of platelets started about 1.600° C.

K. Tsukuma and T. Takahata, (Mat. Res. Soc. Syp. Porc., Vol. 78 (1987), 123-135) disclose a composition of material: ZrO₂ (2 Mol-% (≈3.6 wt-%)Y₂O₃), A1₂0₃ and La₂O₃ and disclose i.a. in table 2 40 wt-% LaAl₁₁O₁₈. The formation occurs at 1.450° C.; the preparation of samples is performed with sintering at 1.500° C., followed by an hot isostatic pressing process also performed at 1.500° C. The Y-TZP/β-LaAl₂O₃-mixture is not so deformable as a Y-TZP/Al₂O₃-mixture and based on this result it can be suggested that platelets take care for the suppression of a plastic deformation (p. 133). Plastic deformation and fracture toughness are in a direct relation. To the skilled person these results do not suggest any relation between platelet formation and increase of fracture toughness.

K. Tsukuma (J. Am. Ceram. Soc., 83(200), 3219-3221) discloses the system Y-TZP:CeO₂:Al₂O₃ in a composition of 60:9,05:30,95. A formation of platelets during sintering in oxidizing atmosphere does not take place. During sintering in reducing atmosphere a new platelet formation can be monitored at a temperature of 1.400° C. At a temperature of 1.500° C. in reducing atmosphere a platelet formation can be monitored. In the mechanical characterization the platelet containing material does not differ substantially from the material Y-TZP/Al₂O₃ so that the author concludes: “The high-temperature bending strength of the converted β-Al₂O₃ composite was almost the same as that of the β-Ce₂O₃ 11Al₂O₃ composite”. Also these experiments teach the skilled person that there is no relation between increasing fracture toughness and platelet reinforced ceramics. Furthermore, no lanthanoxide is used.

The fracture toughness of the Y-TZP materials is still too low today for many applications.

The object of the invention is to provide a material having an improved hydrothermal resistance, high strength and fracture toughness. This object is achieved by the material according to the invention.

The material according to the invention comprises:
a) from 98-50% by volume of zirconia as a matrix,
   i) stabilized with a stabilizing composition having
   ii) of from about 2 to about 3 mole percent of yttria and of from about 10 to about 15 mole percent of ceria; wherein the term mole percent is related to the zirconia matrix and
   iii) the stabilizing composition is present in the range of from about 1:99 to about 99:1; and
b) from about 2 to about 50% by volume of alumina.

In particular the alumina is from about 5 to about 90% by volume in the form of hexagonal platelets of general formula REAl₁₁O₁₈.

The symbol RE means one or more representatives of rare earth metals.

The material according to the invention has a high hydrothermal stability.

In one embodiment, the hexagonal platelets of the material according to the invention may contain lanthanum oxide.

In its chemical composition, the material according to the invention is based on a tetragonally stabilized zirconia matrix. Homogeneously distributed globular alumina particles are incorporated into this matrix. Part of these particles react with the platelet-forming rare earth oxide during the sintering process to form hexagonal platelets of general formula REAl₁₁ O₁₈. The aspect ratio of these hexagonal platelets is at least 2. The abundance of the platelets relative to globular alumina in the zirconia matrix is controlled by the alloyed amount of alumina and rare earth oxide.

The material according to the invention can be prepared by a process comprising the following steps:
grinding the powder mixture in aqueous suspension;
adding a binder;
eliminating coarse particles;
spray-drying;
pressing;
sintering.

A preferred form of the sintering process is hot isostatic postcompaction. When this process is applied, the compact is presintered at first to a density at which a closed porosity is reached. The thus presintered compact is subsequently subjected to a second temperature treatment, an isostatic pressure of from 1 to 150 MPa acting on the component during such temperature treatment. This process step is followed by a further temperature treatment under normal pressure in order to release any residual stress.

Alternatively to pressing, the material may also be admixed with organic auxiliaries in order to become flowable at higher temperatures. This flowable composition is processed by the injection molding method.

The material according to the invention is particularly suitable for preparing ceramic compacts that can be employed in many technical fields.

The ceramic compact according to the invention is obtainable by sintering the material according to the invention.

The sintered compacts according to the invention are characterized by a high mechanical strength of ≧800 MPa as measured according to DIN EN ISO 6872, a high fracture toughness of ≧6 MPa m^{1/2} as measured according to DIN CEN/TS 14425-5, a modulus of elasticity of ≦250 GPa as measured according to DIN EN 843 Part 2 and a Vickers hardness HV_{0.5}≦1500 as measured according to DIN 50113.

The invention also relates to a process for preparing a ceramic compact according to the invention by sintering the material according to the invention.

Due to its low modulus of elasticity and its improved fracture toughness, the compact according to the invention can be employed in the medical field as a high strength and tough material for bridges in the orthodontic field, as a dental implant, as a hip, knee, shoulder, ankle and finger implant.

In engineering, in the mechanical field, the compact according to the invention can be employed, in particular, as a wear component with sealing properties and a high damage tolerance due to its high mechanical strength and its low, for ceramic materials, modulus of elasticity.

The invention is further illustrated by the following Examples.

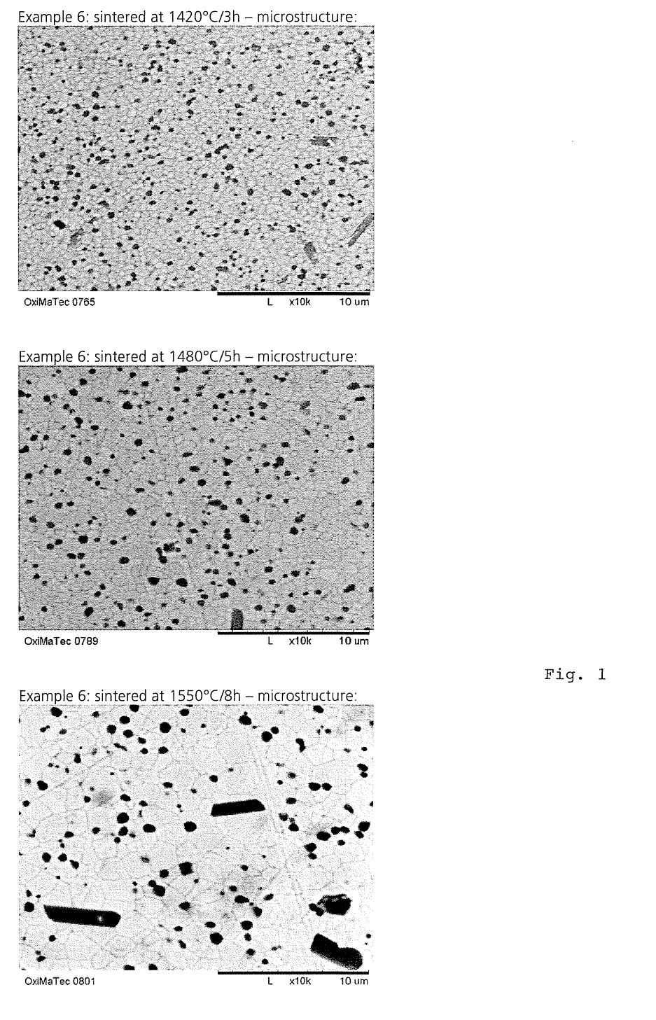

FIG. 1 shows micrographs of the composition of example 6 after treatment at different conditions, sintering temperature at 1420 for 3 h, sintering temperature at 1480 for 5 h, and sintering temperature at 1550 for 8 h.

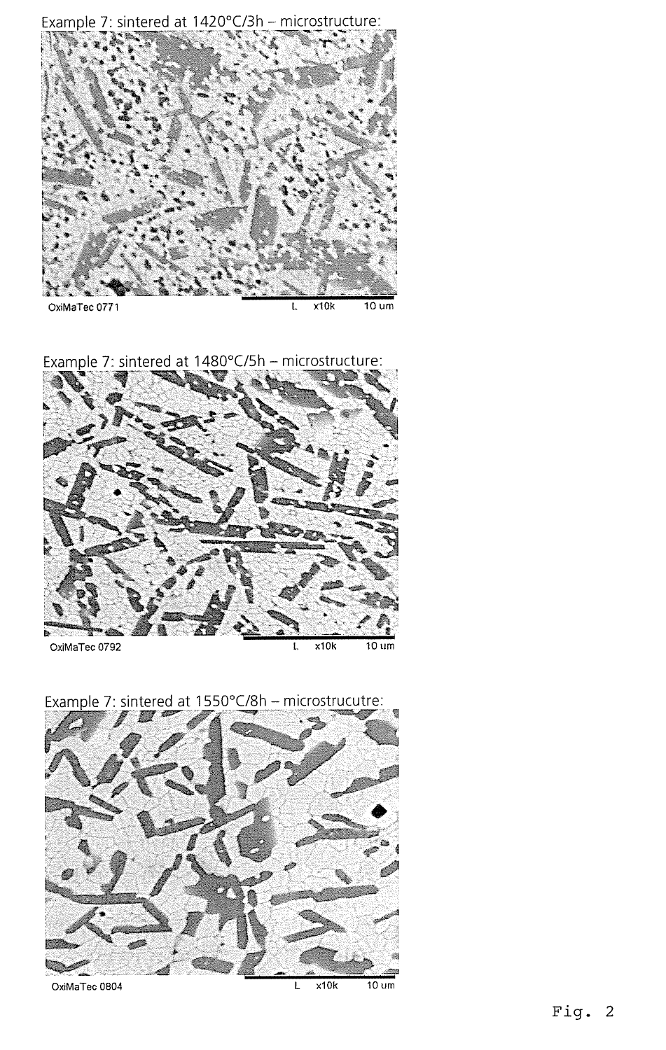

FIG. 2 shows micrographs of the composition of example 7 after treatment at different conditions, sintering temperature at 1420 for 3 h, sintering temperature at 1480 for 5 h, and sintering temperature at 1550 for 8 h.

### EXAMPLES 1-7

The material mixtures summarized in Table 1 were first dispersed in water, and the suspension obtained was subsequently deagglomerated and homogenized in a mixed grinding operation. After having been separated from the milling balls, the ground suspension was admixed with a temporary binder. Subsequently, ready-to-press granules were prepared by applying spray-drying technology. From these ready-to-press granules, specimens were pressed and sintered without pressure.

TABLE 1

| | Ex. 1 | Ex. 2 | Ex. 3 | Ex. 4 | Ex. 5 | Ex. 6 | Ex. 7 |
|---|---|---|---|---|---|---|---|
| ZrO₂ [% by weight] | 59.2 | 82.0 | 80.0 | 74.0 | 86.8 | 84.5 | 67.0 |
| Y₂O₃ [% by weight] | 1.0 | 3.0 | 1.7 | 3.0 | 4.0 | 1.9 | 2.0 |
| CeO₂ [% by weight] | 11.1 | 5.0 | 8.3 | 3.0 | 4.2 | 8.6 | 6.0 |
| Al₂O₃ [% by weight] | 25.0 | 8.5 | 8.3 | 18.6 | 4.5 | 4.5 | 20.0 |
| La₂O₃ [% by weight] | 3.7 | 1.5 | 1.7 | 1.4 | 0.5 | 0.5 | 5.0 |

1. A material comprising:
a) from 98-50% by volume of zirconia as a matrix,
i) stabilized with a stabilizing composition having
ii) of from about 2 to about 3 mole percent of yttria and of from about 10 to about 15 mole percent of ceria; wherein the term mole percent is related to the zirconia matrix and
iii) the stabilizing composition is present in the range of from about 1:99 to about 99:1; and
b) from about 2 to about 50% by volume of alumina.

2. The material according to claim 1, wherein about 5 to about 90% by volume is in the form of hexagonal platelets of general formula REAl₁₁O₁₈.

3. The material according to claim, wherein said hexagonal platelets contain lanthanum oxide.

4. The material according to claim 3, wherein the aspect ratio of said hexagonal platelets is at least 2.

5. A process for preparing a composition comprising providing the material according to claim 1 as a powder mixture wherein the material is further ground in aqueous suspension, admixed with a binder, spray-dried, pressed and sintered.

6. The process according to claim 5, wherein said sintering comprises:
presintering to a density at which a closed porosity is reached, followed by a hot isostatic postcompaction process.

7. The process according to claim 5 wherein the spray-dried powder mixture is further subjected to plasticization, injected into a mold, or freed from binder.

8. A ceramic compact obtainable by sintering the material claim 1.

9. A compact according to claim 8, having a mechanical strength of ≧800 MPa as measured according to DIN EN ISO 6872.

10. The compact according to claim 8 having a fracture toughness of ≧6 MPa·m^{1/2} as measured according to DIN CEN/TS 14425-5.

11. The compact according to claim 8 having a modulus of elasticity of ≦250 GPa as measured according to DIN EN 843 Part 2.

12. The compact according to claim 8 having a Vickers hardness HV_{0.5} of ≦1500.

13. A process for preparing a ceramic compact according to claim 8 by sintering the material claim 1.

14. (canceled)

15. A method of treating a patient comprising:
providing a medical implant comprising the compact of claim 8 for introduction into a patient, wherein the implant is a member of the group consisting of an implant for dental-prosthetic care, a dental implant, a hip implant, a knee implant, a shoulder implant, an ankle implant, and a finger implant.

16. An apparatus comprising:
a medical device comprising a member of the group consisting of a tool for inserting implant screws in the dental field, drill, scalpel, broaching tool and cutter or
a device in the mechanical field in hydraulics and pneumatics, as a wear component with sealing properties, as a printing plate, as a heat-insulating component, as a technical cutting edge and as a non-lubricated slide pairing in microprecision technology.

17. (canceled)

18. (canceled)
